(19) United States
(12) Patent Application Publication
(10) Pub. No.: US 2023/0116332 A1
(43) Pub. Date: Apr. 13, 2023

(54) SYSTEM AND METHOD FOR DIAGNOSING MUSCLE AND BONE RELATED DISORDERS

(71) Applicant: Rajesh Patil, Frisco, TX (US)

(72) Inventor: Rajesh Patil, Frisco, TX (US)

(21) Appl. No.: 17/498,454

(22) Filed: Oct. 11, 2021

Related U.S. Application Data
(63) Continuation-in-part of application No. 17/336,557, filed on Jan. 1, 0001, now abandoned.

(51) Int. Cl. G16H 30/40; G16H 50/20; G16H 20/40; G06N 3/04

(52) U.S. Cl. CPC G16H 30/40; G16H 50/20; G16H 20/40; G06N 3/04

(57) ABSTRACT

Artificial Intelligence Multiple Input Convolutional Neural Network-based system and method to diagnose bone, muscle, or joint diseases using one or multiple inputs such as EMG (Electromyography), X-Ray, MRI (Magnetic resonance imaging), CT (computed tomography), Arthroscopy, Ultrasonography, video, images, patient reports, text reports, The system can provide recommendations or treatment plan based on severity or grading of the disease, deformity or degeneration that may include physiotherapy, exercise, surgery to prevent or cure the medical condition.

### CROSS-REFERENCE TO RELATED APPLICATIONS

This application is a continuation-in-part of a U.S. patent application Ser. No. 17/336,557, filed on Jun. 2, 2021, which is incorporated herein by reference in its entirety.

### FIELD OF INVENTION

The present invention relates to an Artificial Intelligence—Multi-input Convolutional Neural Network based system and method for diagnosing and providing recommendations to prevent or treat a medical condition, and more particularly, the present invention relates to a system and method for diagnosing bone and muscle related disorders using Artificial Intelligence—Multi-Input Convolutional Neural Networks.

### BACKGROUND

Muscle and joint-related medical conditions have increased several folds in the last decade. The primary reason is the unhealthy lifestyle and food habits. However, there could be many other reasons, such as genetic, weight, physical activity, sports, age, gender, injury, and like. In general, the primary causes for the said medical condition include the excessive extension of the joint and weakness of muscles around the joint.

The current method for detecting the bone, muscle, or joint deformity or disorder uses various screening techniques, such as X-ray, MRI (Magnetic Resonance Imaging), CT (Computed Tomography) scan, Ultrasonography, and Arthroscopy. However, the known diagnostic methods suffer from one or more drawbacks including the low level of accuracy in diagnosis and human subjectivity. Inaccurate diagnosis of the medical condition leads to ineffective treatment and often worsening of the medical conditions.

Thus, a desire is there for a system and method that can accurately diagnose a medical condition related to bones and muscles degeneration or deformity.

Hereinafter, the terms “disorder”, “disease”, “degeneration”, and “deformity” will be interchangeably used and used in relation to a medical condition of bone, muscle, and/or joints.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system and method for diagnosing a medical condition related to bone and muscles.

It is another object of the present invention that the system and method can recommend a suitable treatment.

It is still another object of the present invention that the diagnosis is fairly accurate.

It is yet another object of the present invention that the human intervention is nil or minimum in the diagnosis.

In one aspect, disclosed is a medical diagnosis system for diagnosing a disorder and/or severity of the disorder related to a bone and/or muscle, the medical diagnosis system includes a processor and a memory configured to implement the steps of receiving two or more types of medical imaging data relating to a body part of a patient as an input; pre-processing the input by using normalization and standardization; and implement a multi-input convolutional neural network (MI-CNN), the MI-CNN comprising a plurality of processing layers in an order, each layer of the plurality of processing layers comprises at least one node, wherein at least one node of each layer is connected to at least one node of a following layer or a preceding layer in the order, the plurality of processing layers includes a convolution layer for feature extraction from an image, a rectified linear activation function to scale data into a linear scale of 0 and 1, a pooling layer for dimension reduction extracting dominant features, suppressing noise, increasing computational power and analysis accuracy, a flatten layer to convert the pooling layer to a single column vector, and a full connection layer to compute class scores and classify features for meaningful output deviation; and applying the MI-CNN model to the pre-processed input for grading the severity of the disorder to obtain a grade.

In one implementation of the medical diagnosis system, the processor and memory are further configured to present the grade and a recommendation for treatment based on the grade. The medical imaging data can be obtained from medical imaging modalities selected from a group consisting of Electromyography, X-Ray, Magnetic resonance imaging, Computed tomography, Ultrasonography, Arthroscopy, or a combination thereof. In one case, the body part can be a knee and the disorder can be patellofemoral pain syndrome. The input can further include a patient's data selected from a group consisting of weight, age, body mass index, or a combination thereof.

In one aspect, disclosed is a method for diagnosing a disorder and/or severity of the disorder related to a bone and/or muscle, the method implemented within a medical diagnosis system, the method includes the steps of receiving two or more types of medical imaging data relating to a body part of a patient as an input; pre-processing the input by using normalization and standardization; and implementing a multi-input convolutional neural network (MI-CNN), the MI-CNN comprising a plurality of processing layers in an order, each layer of the plurality of processing layers comprises at least one node, wherein at least one node of each layer is connected to at least one node of a following layer or a preceding layer in the order, the plurality of processing layers includes a convolution layer for feature extraction from an image, a rectified linear activation function to scale data into a linear scale of 0 and 1, a pooling layer for dimension reduction extracting dominant features, suppressing noise, increasing computational power and analysis accuracy, a flatten layer to convert the pooling layer to a single column vector, and a full connection layer to compute class scores and classify features for meaningful output deviation; and applying the MI-CNN model to the pre-processed input for grading the severity of the disorder to obtain a grade.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a block diagram showing the system architecture, according to an exemplary embodiment of the present invention.

FIG. 2 shows an exemplary embodiment of the multi-input CNN model, according to the present invention.

FIG. 3 is a flow chart showing a method for diagnosing a disorder using the multi-input CNN model, according to an exemplary embodiment of the present invention.

FIG. 4 shows a medical report as an output of the multi-input CNN model, according to an exemplary embodiment of the present invention.

FIG. 5 shows an MRI report as an input for the multi-input CNN model, according to an exemplary embodiment of the present invention.

FIG. 6 shows an X-ray report as an input for the multi-input CNN model, according to an exemplary embodiment of the present invention.

FIG. 7 shows an EMG report as input for the multi-input CNN model, according to an exemplary embodiment of the present invention.

FIG. 8 shows an arthroscopy report as input for the multi-input CNN model, according to an exemplary embodiment of the present invention.

FIG. 9 shows an exemplary embodiment of the EMG data that can be fed to the MI-CNN, according to an exemplary embodiment of the present invention.

FIG. 10a illustrates preprocessing steps for MI-CNN of Electromyography raw-walking-data of a patient.

FIG. 10b illustrates the preprocessing steps for MI-CNN after the Normalization of data.

FIG. 10c illustrates the preprocessing steps for MI-CNN after the standardization of data.

FIG. 10d illustrates the preprocessing steps for MI-CNN data after Normalization and Standardization.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

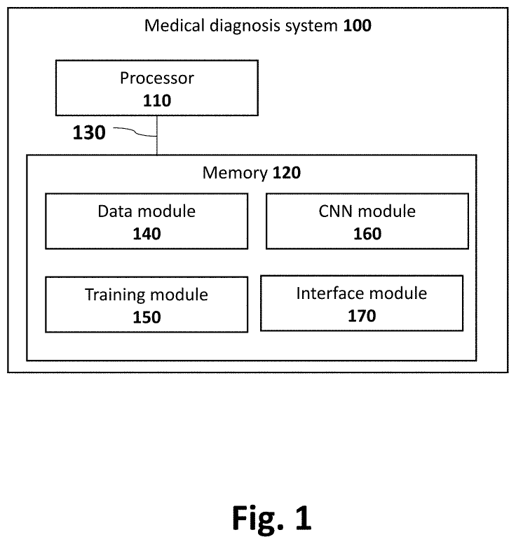

Disclosed is a multi-input Convolutional Neural Network (MI-CNN) based system and method for diagnosing a disorder of muscles and bones based on automated analysis of medical imaging data and recommending treatment for same. Referring to FIG. 1, which is a block diagram of the disclosed medical diagnosis system 100 that can include a processor 110 and a memory 120, wherein the processor 110 can be coupled to the memory 120 through a system bus 130. The processor 110 can be any logic circuitry that responds to and processes instructions fetched from the memory 120. Suitable examples of the processors commercially available are Intel and AMD. The memory 120 may include one or more memory chips capable of storing data and allowing any storage location to be directly accessed by the processor 110. As shown in FIG. 1, the memory 120 includes modules in accordance with an exemplary embodiment of the present invention for execution by the processor 110 performing one or more steps of the disclosed method for diagnosing muscle and bone disorders. The memory 120 can include a data module 140, a training module 150, a CNN module 160, and an interface module 170. The data module 140 upon execution by the processor can receive input data of the patient for which diagnose has to be performed. In one case, the data module 140 can receive medical imaging data, such as but not limited to EMG (Electromyography), X-Ray, MRI (Magnetic resonance imaging), CT (computed tomography), Ultrasonography, Arthroscopy, medical imaging report in the form of video or set of images, patient medical reports, and like. Examples of videos include videos of patient MRI report data that is dynamic, which include videos of bone, muscles, tendons, blood vessels, and internal organs of joints. In one case, selected frames in the video can be combined to form a composite image. In one case the medical imaging data can be images captured by the medical imaging device that can be representative of parts of the body showing the internal structures for medical diagnosis.

The training module 150 upon execution by the processor can provide training of the CNN module wherein the training module can use different types of medical imaging data, such as but not limited to EMG (Electromyography), X-Ray, MRI (Magnetic resonance imaging), CT (computed tomography), Ultrasonography, Arthroscopy, medical imaging video and images, patient medical reports, and like, as the training dataset.

The CNN module 160 upon execution by the processor can provide for grading the severity of the disorder and provide a recommendation for the treatment. The CNN module 160 can take multiple inputs, such as but not limited to combinations of one or more medical imaging technologies including EMG (Electromyography), X-Ray, MRI (Magnetic resonance imaging), CT (computed tomography), Ultrasonography, Arthroscopy, imaging data in the form of video and images, patient medical reports, and like for grading the severity of the disorder. The recommendations can include exercises, physiotherapy, surgery, and known treatments.

The interface module 170 upon execution by the processor may provide an interface for interacting with the disclosed medical diagnosis system 100. For example, a screen can be provided to input the different types of medical data of the patient. Similarly, the determined medical condition and the treatment plan can also be displayed on a screen of the interface.

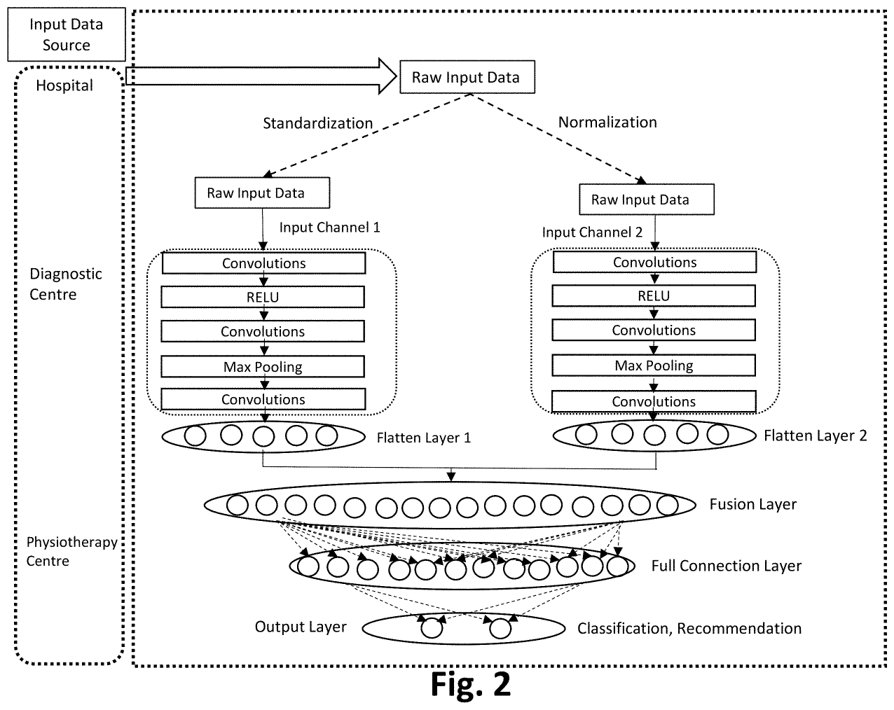

Referring to FIG. 2 which shows an exemplary embodiment of the deep learning neuronal network. Raw input data of a patient can be obtained from different sources, such as hospitals, diagnostic centers, and physiotherapy centers. For example, the patient medical history can be obtained from the hospital while the medical imaging data, such as X-rays can be obtained from diagnostic centers. The data pre-processing step includes data scaling which can be achieved by normalization and standardization. Standardization rescales data to have a mean of 0 and a standard deviation of 1. Normalization can rescale the values into a range of [0, 1]. The data can be cleaned, augmented by horizontally flipping, wherein only that data can be ingested that is within defined entry criteria (resolution, pixel) or else can reject for the multi-dimensional time-series signal to prevent data overfitting and improve model accuracy.

The CNN can include fully connected processing layers wherein the input layer nodes are connected to every node in the following layer. A number of nodes can be present in each layer and any number of such nodes are within the scope of the present invention. In one case, the number of nodes can be limited to prevent overfitting. For example, the number of nodes can be at least one of the number of nodes can be up to 5 or 10. The convolution layer can provide for feature extraction from the original image (reduced/increased) in dimensionality, enhancing the accurate analysis of images. Rectified linear activation function (ReLU) can be an activation function to scale data into a linear scale of 0 and 1 and can converge data quickly. The pooling layer provides for dimension reduction extracting dominant features, suppresses noise, increases computational power and analysis accuracy. Flatten layer can convert the multidimensional pooling layer to a single column vector. The fusion layer includes a model compression technique that can discover if which weights to combine and then fuses weights of similar fully connected. The full connection layer can compute class scores and classify the features for meaningful output deviation—the grade of medical condition (this is further fed to the recommendation system for remedial action). The output of the CNN can be the classification and the recommendation.

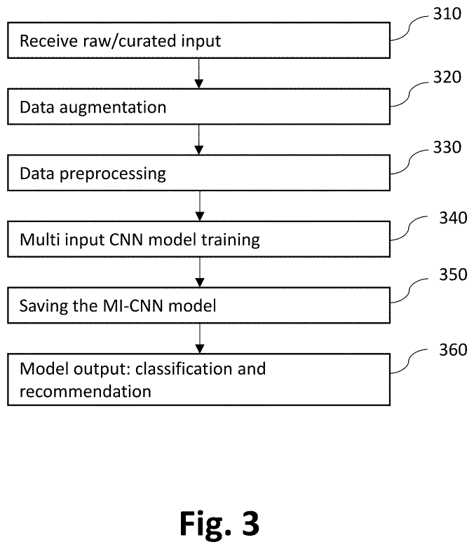

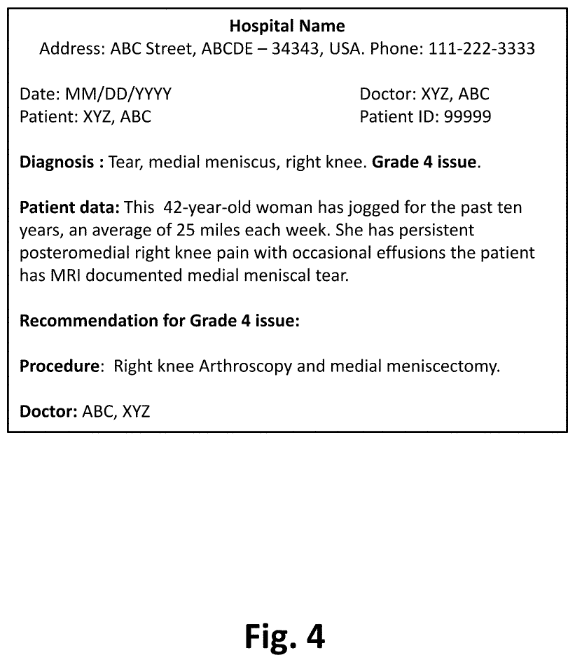

Referring to FIG. 3, which is a flowchart showing an exemplary embodiment of the disclosed deep learning neuronal network-based method for grading the severity of muscle or bone-related disorder. Raw/curated input related to the patient's medical condition, in particular the medical imaging data, such as EMG, MRI, X-ray, CT-Scan, Ultrasonography, and Arthroscopy can be obtained at step 310. The input data can be augmented, at step 320, and can include the steps of noise reduction, compression, and orientation. Thereafter, the data can be pre-processed, at step 330 and may involve normalization and standardization. The data can be used as a training dataset to train a multi-input CNN model, at step 340. The multi-input CNN model can then be saved, at step 350 and include saving all the CNN parameters. The MI-CNN model can generate output including grade and recommendation, at step 360. Referring to FIG. 4 which shows an exemplary embodiment of the output of the CMI-CNN model. It can be seen that the report shown in FIG. 4 can include the diagnosed grading of disorder, Grade-4 in the report. The patient data can be obtained from patient medical records, also referred to herein as the patient's data. Also, a recommendation has been prescribed based on the grade.

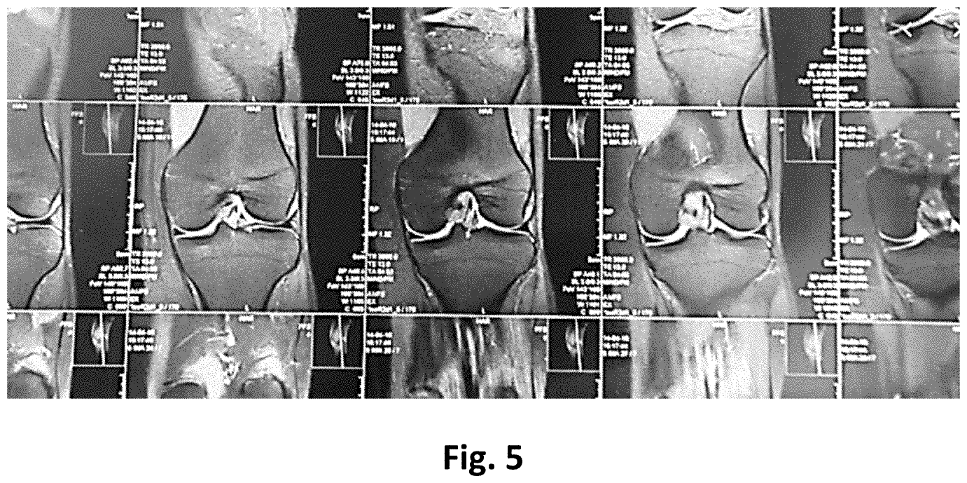

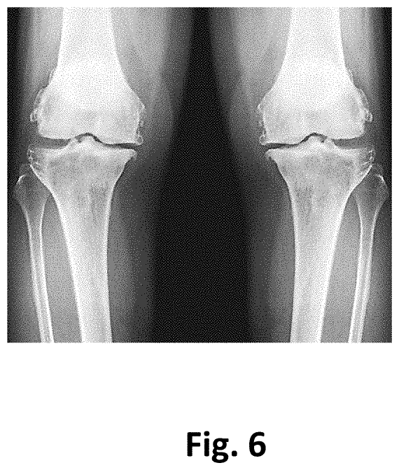

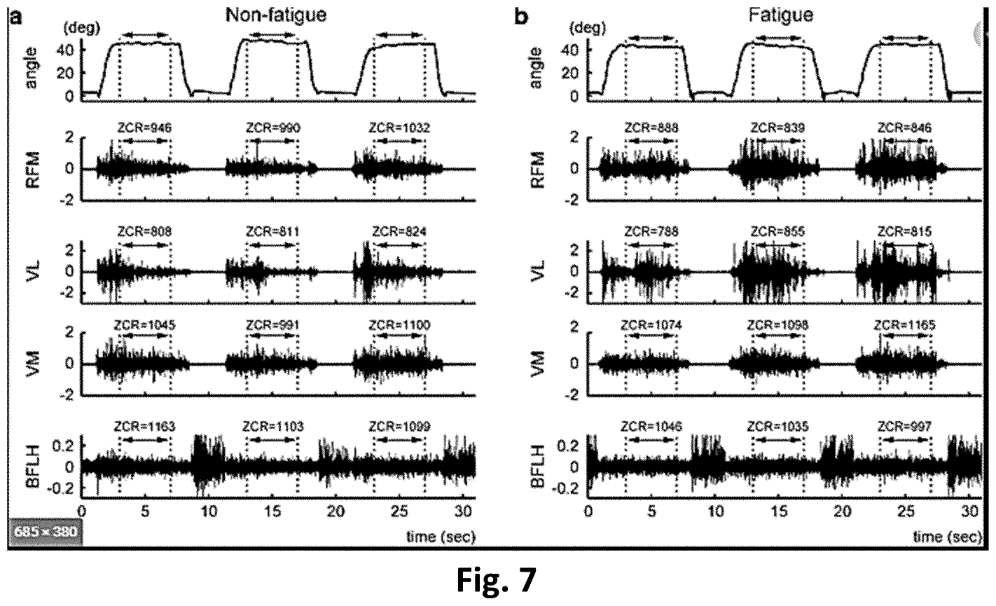

Referring to FIG. 5 shows an example of an MRI scan report that can be taken as an input for the disclosed MI-CNN model. FIG. 6 shows an X-ray report of the knee that can be used as an input for the MI-CNN model. FIG. 7 shows an EMG report for diagnosing Patellar or Knee joint-related issues that can be used as input to automatically extract features to classify patients with an appropriate degree of muscle or bone deformity for patellar issues. Electromyography (EMG) measures muscle response or electrical activity in response to a nerve's stimulation of the muscle. The EMG report shown in FIG. 7 depicts the data of three joint angles (hip flexion angle (HF), knee flexion angle (KF), ankle dorsiflexion angle (ADF) of the lower limbs, and EMG signals of 4 muscles, Rectus femoris (RFM), vastus lateralis (VL), vastus medialis (VM), biceps femoris (BFLH). The non-fatigue data on the left side depicts data when the patient or subject is static, not moving. The fatigue data on the right side depicts data when the patient or subject is walking or running, that is in motion to identify the response and medical condition of joints, bones, muscles.

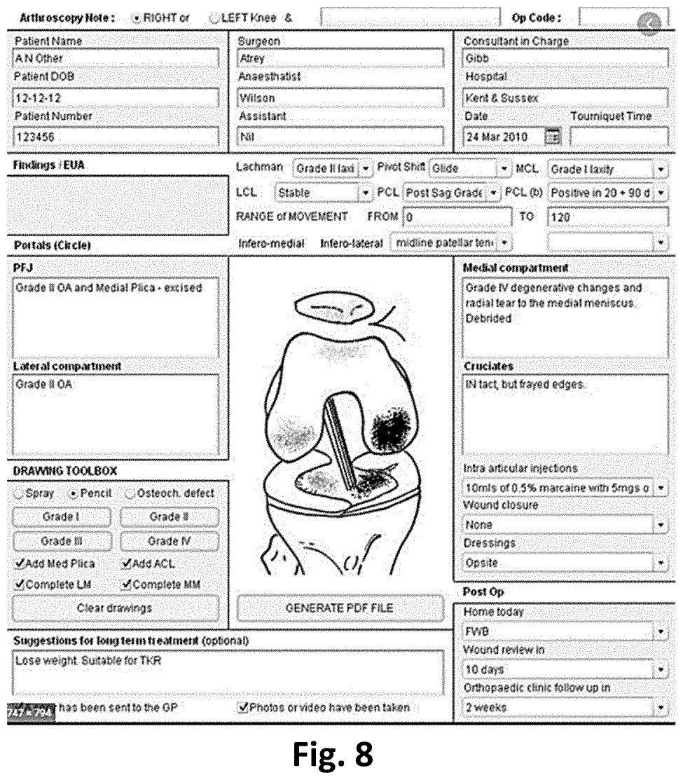

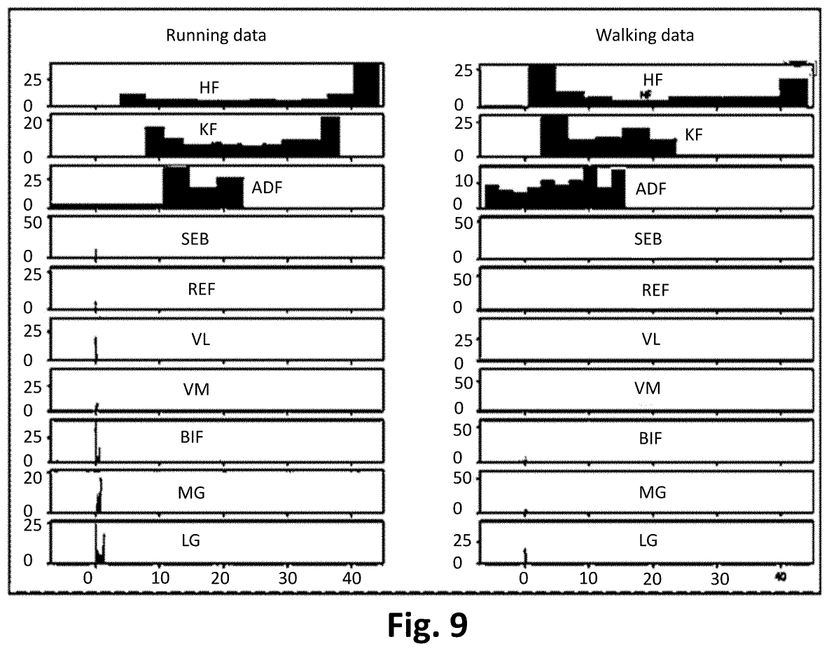

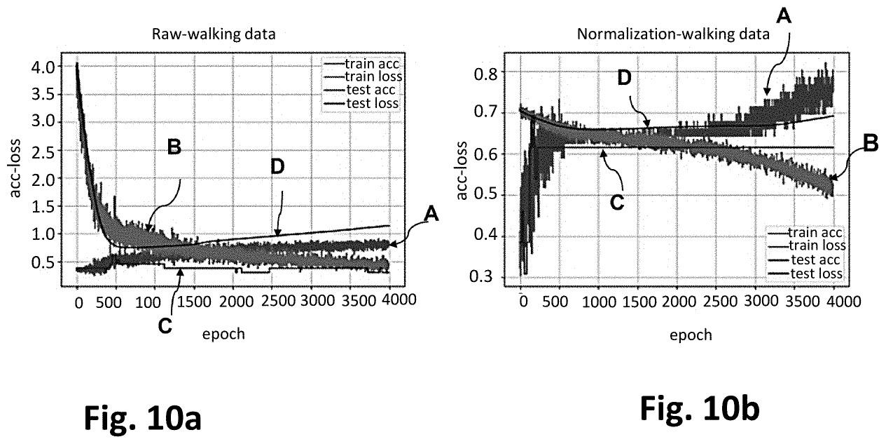

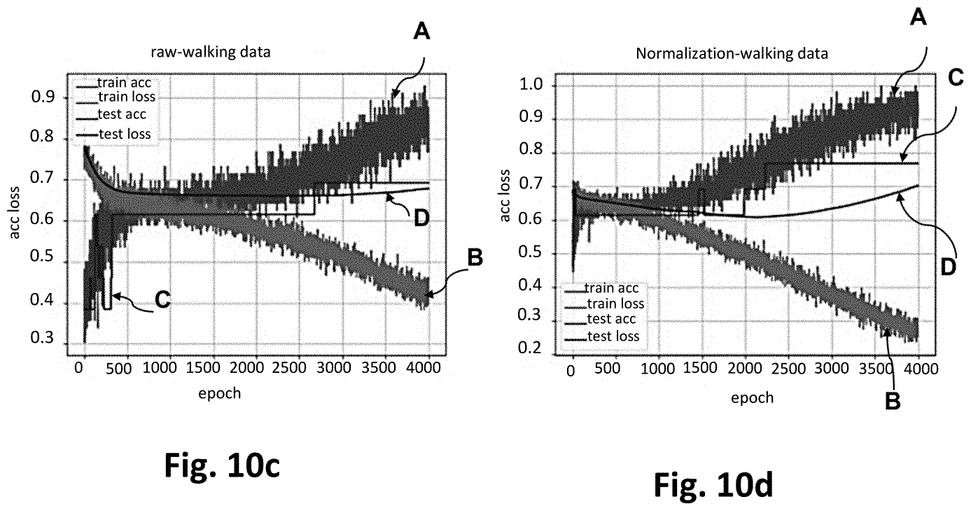

Referring to FIG. 8 which shows an example arthroscopy report of the right knee as the body part and suggesting grade— IV degenerating changes. The arthroscopy report can be appending with images and/or videos of the body part. Referring to FIG. 9 which shows the walking and running EMG data that can be fed to the MI-CNN model. FIG. 10 shows the output of different pre-processing steps for the walking EMG data. FIG. 9(a) shows the Electromyography raw walking data of a patient. FIG. 9(b) shows an output of the normalization step in pre-processing the data. FIG. 9(c) shows an output of the standardization step in pre-processing the data. FIG. 9(d) shows MI-CNN data after Normalization and Standardization that is the input to the Convolutional Neural Network. In the FIGS. 9(a-d), curve A indicates the training accuracy of the MI CNN model, curve B depicts the training loss of MI CNN, curve C depicts test accuracy of MI CNN model, and curve D depicts test loss of MI CNN model.

### Example 1

Patellofemoral pain syndrome (PFPS) is an extremely common medical condition or disease of the knee. Symptoms of PFPS include gradual onset of knee pain behind or around the patella (especially among runners, athletes, middle-aged women, overweight, around age 60).

Pain while climbing up and down the stairs or squatting, eventually evolves into patellofemoral osteoarthritis, if not treated in time, can cause joint deformities and disability. Hence, early, and accurate diagnosis of PFPS is incredibly important. The medical condition can be divided into four grades. Grade 1 severity indicates softening of the cartilage in the knee area. Grade 2 indicates a softening of the cartilage along with abnormal surface characteristics. This usually marks the beginning of tissue erosion. Grade 3 shows thinning of cartilage with active deterioration of the tissue. Grade 4, the most severe grade, indicates exposure of the bone with a significant portion of cartilage deteriorated. Bone exposure means bone-to-bone rubbing is likely occurring in the knee. Currently, the medical condition can be detected by X-ray, MRI magnetic resonance imaging (long, claustrophobia, misdiagnosis by inexperience), computed tomography, arthroscopy (high accuracy but invasive). The specific root cause of the disorder and degree of severity is difficult to detect and remains unclear. As the root cause and the degree of severity remain unclear, the physiotherapists and doctors are not able to provide accurate and effective recommendations to patients-physiotherapy or surgery. The disclosed CNN model can provide a solution to aforesaid drawbacks in known diagnostics methods. The multi-input Convolutional neural network model can be used for computer-aided diagnosis, determination of the degree of severity, and recommendation on treatment. Since PFPS involves different biomechanical characteristics of the lower limbs, the disclosed CNN can consider multiple biomechanical characteristics to derive the root cause of PFPS. As the data distribution between different characteristic dimensions can be different, data preprocessing becomes necessary to make the different characteristic dimensions comparable to each other. The disclosed multi-input CNN can use 2 input channels to investigate the information of lower limb biomechanical data, pre-process by two methods (standardization and normalization) to diagnose PFPS. The data can be cleaned, augmented by horizontally flipping, only ingest data that is within defined entry criteria (resolution, pixel) or else reject for the multi-dimensional time-series signal to prevent data overfitting and improve model accuracy. Three joint angles (hip flexion angle (HF), knee flexion angle (KF), ankle dorsiflexion angle (ADF) of the lower limbs and EMG signals of seven muscles (semimembranosus (SEB), rectus femoris (REF), vastus lateralis (VL), vastus medialis (VM), biceps femoris (BIF), medial gastrocnemius (MG), and lateral gastrocnemius (LG) would be used as input to automatically extract features to classify patients with an appropriate degree of PFPS. The disclosed medical diagnosis system can combine the appropriate neural network model and biomechanical analysis to establish accurate, convenient, and real-time diagnosis of PFPS with an accurate degree of severity to prevent misdiagnosis.

Regarding different grades of severity of the PFPS, the disclosed MI CNN model can classify the condition into either of the four grades based on at least two inputs. The MI CNN model can also suggest the recommendation with the classification. For example, for grade 1: Recommend reducing pressure on the kneecap & joint, resting, stabilizing, icing the joint, over-the-counter pain medicines, wearing right shoes and shoe inserts; Grade 2: Grade 1 Recommendation AND Physical therapy focusing on strengthening quadriceps, hamstrings, adductors, abductors to improve muscle strength and balance (Muscle balance will help prevent knee misalignment; Grade 3: Grade 2 Recommendations AND doing non-weight-bearing exercises, such as swimming or riding a stationary bike. Additionally, isometric exercises involving tightening and releasing muscles to help maintain muscle mass; and Grade 4: Grade 3 Recommendations AND Surgery: Arthroscopic surgery may be necessary to examine the joint and determine whether there's a misalignment of the knee. The surgery involves inserting a camera into the joint through a tiny incision. The surgical procedure may fix the problem. Another common procedure is lateral release. This operation involves cutting some of the ligaments to release tension and allow for more movement. Other surgical options may involve smoothing the back of the kneecap, implanting a cartilage graft, or relocating the insertion of the thigh muscle.

In one exemplary embodiment, the MI CNN can classify or grade the deformity as follows: Grade 1 severity indicates softening of the cartilage/muscle/tissue in any joint area; Grade 2: severity indicates a softening of the cartilage/muscle/tissue along with abnormal surface characteristics. This usually marks the beginning of tissue erosion. Grade 3: shows thinning of cartilage/muscle/tissue with active deterioration of the tissue; and Grade 4, the most severe grade, indicates exposure of the bone with a significant portion of cartilage/muscle/tissue deteriorated. Bone exposure means bone-to-bone rubbing is likely occurring in the joint and could lead to some cracks as well.

In one exemplary embodiment, the patient data, such as age, weight, body mass index (BMI) can be used along with the medical imaging data as the input data for the disclosed system.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A medical diagnosis system for diagnosing a disorder and/or severity of the disorder related to a bone and/or muscle, the medical diagnosis system comprising:
a processor and a memory configured to implement the steps of:
receiving two or more types of medical imaging data relating to a body part of a patient as an input;
pre-processing the input by using normalization and standardization; and
implement a multi-input convolutional neural network (MI-CNN), the MI-CNN comprising a plurality of processing layers arranged in an order, each layer of the plurality of processing layers comprises at least one node, wherein at least one node of each layer of the plurality of processing layers is connected to at least one node of a following layer or a preceding layer in the order, the plurality of processing layers comprises:
a convolution layer for feature extraction from an image,
a rectified linear activation function to scale data into a linear scale of 0 and 1,
a pooling layer for dimension reduction extracting dominant features, suppressing noise, increasing computational power, and analysis accuracy,
a flatten layer to convert the pooling layer to a single column vector, and
a full connection layer to compute class scores and classify features for meaningful output deviation; and
applying the MI-CNN to the pre-processed input for grading the severity of the disorder to obtain a grade.

2. The medical diagnosis system according to claim 1, wherein the processor and memory are further configured to:
presenting the grade and a recommendation for treatment based on the grade.

3. The medical diagnosis system according to claim 1, wherein the medical imaging data is obtained from medical imaging modalities selected from a group consisting of Electromyography, X-Ray, Magnetic resonance imaging, Computed tomography, Ultrasonography, Arthroscopy, or a combination thereof.

4. The medical diagnosis system according to claim 3, wherein the body part is a knee.

5. The medical diagnosis system according to claim 3, wherein the disorder is patellofemoral pain syndrome.

6. The medical diagnosis system according to claim 3, wherein the input further comprises a patient's data selected from a group consisting of weight, age, body mass index, or a combination thereof.

7. A method for diagnosing a disorder and/or severity of the disorder related to a bone and/or muscle, the method implemented within a medical diagnosis system, the medical diagnosis system comprising a processor and a memory, the method comprising the steps of:
receiving two or more types of medical imaging data relating to a body part of a patient as an input;
pre-processing the input by using normalization and standardization; and
implementing a multi-input convolutional neural network (MI-CNN), the MI-CNN comprising a plurality of processing layers arranged in an order, each layer of the plurality of processing layers comprises at least one node, wherein at least one node of the each layer is connected to at least one node of a following layer or a preceding layer in the order, the plurality of processing layers comprises:
a convolution layer for feature extraction from an image,
a rectified linear activation function to scale data into a linear scale of 0 and 1,
a pooling layer for dimension reduction extracting dominant features, suppressing noise, increasing computational power, and analysis accuracy,
a flatten layer to convert the pooling layer to a single column vector, and
a full connection layer to compute class scores and classify features for meaningful output deviation; and
applying the MI-CNN to the pre-processed input for grading the severity of the disorder to obtain a grade.

8. The method according to claim 7, wherein the method further comprises the steps of:
presenting the grade and a recommendation for treatment based on the grade.

9. The method according to claim 7, wherein the medical imaging data is obtained from medical imaging modalities selected from a group consisting of Electromyography, X-Ray, Magnetic resonance imaging, Computed tomography, Ultrasonography, Arthroscopy, or a combination thereof.

10. The method according to claim 9, wherein the body part is a knee.

11. The method according to claim 7, wherein the disorder is patellofemoral pain syndrome.

12. The method according to claim 7, wherein the input further comprises a patient's data selected from a group consisting of weight, age, body mass index, or a combination thereof.
